(19) United States
(12) Patent Application Publication
(10) Pub. No.: US 2015/0061679 A1
(43) Pub. Date: Mar. 5, 2015

(54) Patient Support Apparatus

(71) Applicants: Yvonne Candidus, Furth (DE); Daniel Driemel, Oederan (DE); Hubertus Fischer, Bamberg (DE); Wolfgang Kraus, Furth (DE); Thomas Kundner, Buckenhof (DE); Martin Zigann, Brunn (DE); Stephan Zink, Erlangen (DE)

(72) Inventors: Yvonne Candidus, Furth (DE); Daniel Driemel, Oederan (DE); Hubertus Fischer, Bamberg (DE); Wolfgang Kraus, Furth (DE); Thomas Kundner, Buckenhof (DE); Martin Zigann, Brunn (DE); Stephan Zink, Erlangen (DE)

(21) Appl. No.: 14/476,472

(22) Filed: Sep. 3, 2014

(30) Foreign Application Priority Data
Sep. 3, 2013 (DE) 102013217539.6

(51) Int. Cl. A61B 5/055; G01R 33/38; G01R 33/48; A61G 1/048

(52) U.S. Cl. CPC A61B 5/0555; A61G 1/048; G01R 33/38; G01R 33/48
USPC 324/321; 5/81.1 R

(57) ABSTRACT

A patient support apparatus configured to support a patient is provided. The patient support apparatus includes a table having a support surface to support the patient. The table includes a patient support assistance unit with at least one handle element.

This application claims the benefit of DE 10 2013 217 539.6, filed on Sep. 3, 2013, which is hereby incorporated by reference in its entirety.

### BACKGROUND

The support and/or positioning of patients (e.g., physically impaired patients) on a medical patient support apparatus requires major expenditure of force on the part of medical operators (e.g., physicians or medical technical assistants). For example, the change of support and/or repositioning from a sitting position to a horizontal position and vice versa imposes a major strain on medical operators.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, support and/or a change of support for a patient on a patient support apparatus assisted and/or facilitated.

A patient support apparatus configured to support a patient is provided. The patient support apparatus includes a table having a support surface to support the patient.

The table includes a patient support assistance unit with at least one handle element. The patient support assistance unit is configured to assist a support operation (e.g., a change of support operation and/or a repositioning operation from a sitting position to a horizontal position or from a horizontal position to a sitting position for the patient). The patient support assistance unit (e.g., the handle element of the patient support assistance unit) may be configured to provide support for the patient. The patient is able to use his hand to support himself on the patient support assistance unit (e.g., on the handle element of the patient support assistance unit) during a change of support and/or repositioning. This allows a change of support and/or repositioning of the patient to be assisted and/or facilitated in an advantageous manner. Also, the force expenditure on the part of a medical operator during the change of support and/or repositioning of the patient may be minimized. The patient support assistance unit (e.g., the handle element of the patient support assistance unit) also allows a high level of safety to be achieved during the change of support and/or repositioning of the patient, thereby making the patient feel extremely safe.

In addition to the table, the patient support apparatus also includes a base unit. The table is supported such that the table may be moved in relation to the base unit. The patient support apparatus may have a sliding bearing unit.

The at least one handle element may be arranged at least partially in a center region of the table. This makes the at least one handle element advantageously accessible for the patient during a change of support operation and/or a repositioning operation. The at least one handle element may, for example, be arranged in the region of the patient's hip on the table. The at least one handle element is, for example, arranged in a center region on the table in the direction of a longitudinal extension of the table.

The patient support assistance unit may be formed by a separate unit configured solely to provide support for the patient during a support operation (e.g., a change of support operation and/or a repositioning operation from a sitting position to a horizontal position or from a horizontal position to a sitting position) for the patient. Alternatively or additionally, the patient support assistance unit may also be integrated at least partially in a securing unit arranged at the side (e.g., a securing grid element) of a mobile patient support apparatus.

A particularly space-saving arrangement of the patient support assistance unit (e.g., the handle element of the patient support assistance unit) may advantageously be achieved if the at least one handle element is arranged in a peripheral region of the table. This provides, for example, that a region of the patient support apparatus configured to hold the patient may remain unrestricted. In this context, a peripheral region refers, for example, to a region of the table that may be arranged in proximity to an edge and outside a support region for supporting the patient.

If the at least one handle element is arranged on a side of the table, the side of the table being aligned perpendicular to the support surface, the patient support assistance unit (e.g., the handle element of the patient support assistance unit) may be integrated within the patient support apparatus such that a patient support region remains available in an unrestricted manner to support the patient.

In one embodiment, the at least one handle element is supported on the table such that the at least one handle element may be moved between a first grip position and at least one second grip position. The patient support assistance unit (e.g., the handle element of the patient support assistance unit) may thus advantageously be adjusted for different situations (e.g., an examination situation and/or a support situation), so that there is no undesirable obstruction of the patient and/or of the examination. Also, for a further positioning operation for the patient on the patient support apparatus (e.g., for the positioning of additional units; an infusion unit and/or local magnetic resonance coil units, etc.), the handle element may be moved to a grip position that does not obstruct the further positioning operation. The at least one handle element may be supported on the table such that the at least one handle element may be folded and/or rotated between at least two grip positions.

A space-saving and compact arrangement of the patient support assistance unit (e.g., the handle element of the patient support assistance unit) within the patient support apparatus may be achieved if the table has a thickness, and the at least one handle element is arranged within the thickness in a first grip position on the table. The first grip position may include a passive grip position, so that in this first grip position, the patient support assistance unit (e.g., the handle element of the patient support assistance unit) is arranged so that the patient support assistance unit is folded in against the support table. The patient support assistance unit (e.g., the handle element of the patient support assistance unit) is also configured not to be actuated and/or to provide support for a patient in this first and/or passive grip position. In this first and/or passive grip position of the patient support assistance unit (e.g., the handle element of the patient support assistance unit), the patient and the patient support apparatus are in an examination position (e.g., within a patient receiving region of a medical imaging apparatus), or the patient leaves the patient support apparatus, so that such operations are not obstructed by the patient support assistance unit (e.g., the handle element of the patient support assistance unit). In this first grip position, the handle element may also be arranged completely within the thickness of the table.

In a further embodiment, the at least one handle element projects into a patient support region of the table in a second grip position on the table. The second grip position may include an active grip position, in which the patient support assistance unit (e.g., the handle element of the patient support assistance unit) is configured to assist a support operation (e.g., a change of support operation and/or a repositioning operation from a sitting position to a horizontal position or from a horizontal position to a sitting position) for the patient. The patient support region of the table may include the support surface of the table and a region adjoining the support surface, in which the patient positioned and/or supported on the support surface at least partially rests.

Simple operation and/or accessibility of the at least one handle element may advantageously be achieved for a patient if the at least one handle element has a longitudinal extension that is inclined at an angle of between at least 45° and maximum 90° to the support surface of the table in the second position on the table. The angle of inclination also allows support to be provided for the patient in an advantageous manner to assist a support operation (e.g., a change of support operation and/or a repositioning operation from a sitting position to a horizontal position or from a horizontal position to a sitting position) for the patient.

In one embodiment, the at least one handle element has a longitudinal extension, whereby the longitudinal extension of the handle element is aligned essentially parallel to a longitudinal extension of the table when the handle element is in the second grip position on the table. Simple operation and/or accessibility of the at least one handle element may thus be achieved for a patient. Essentially parallel may, for example, be that the longitudinal extension of the handle element is at an angle between 150° to 210 (e.g., an angle between 160° to 200° or an angle between 170° to 190°) to the longitudinal extension of the table.

In a further embodiment, the table and/or the patient support assistance unit has a setting unit configured to set a grip position of the at least one handle element on the table. This allows simple setting and/or advantageous securing of the grip position to be achieved (e.g., while the handle element is loaded). The setting unit may, for example, include a sliding bearing unit and/or a latching unit and/or further units suitable for setting and/or securing the grip position of the handle element.

If the patient support apparatus has a sensor unit configured to detect a grip position of the at least one handle element on the table, a high safety standard may be achieved for the patient support apparatus. An unwanted collision of the patient support assistance unit (e.g., the handle element of the patient support assistance unit) with, for example, a housing enclosing a patient receiving region of a medical imaging apparatus as the table is introduced into the patient receiving region of the medical imaging apparatus may thus be prevented.

In one embodiment, a medical imaging apparatus with a magnet unit, a patient receiving region at least partially enclosed by the magnet unit and a patient support apparatus with a patient support assistance unit is provided. The patient support assistance unit (e.g., the handle element of the patient support assistance unit) may be configured to provide support for the patient. The patient is able to support him/herself using his/her hand on the patient support assistance unit (e.g., on the handle element of the patient support assistance unit) during the change of support and/or repositioning This allows a change of support and/or repositioning of the patient to be advantageously assisted and/or facilitated. Also, the force expenditure on the part of the medical operator during the change of support and/or repositioning of the patient may be minimized. Also, the patient support assistance unit (e.g., the handle element of the patient support assistance unit) allows a high level of safety to be achieved during the change of support and/or repositioning of the patient, thereby making the patient feel extremely safe.

In one embodiment, the medical imaging apparatus has a monitoring unit configured to monitor a grip position of at least one handle element of the patient support assistance unit. The monitoring unit may include a sensor unit for detecting the grip position of the handle element. A high safety standard for the patient on the patient support apparatus may be achieved. An unwanted collision of the patient support assistance unit (e.g., the handle element of the patient support assistance unit) with, for example, a housing enclosing a patient receiving region of a medical imaging apparatus as the table is introduced into the patient receiving region of the medical imaging apparatus may thus be prevented. The monitoring unit may also be arranged at least partially within the patient support apparatus and/or within the patient support assistance unit.

In one embodiment, the monitoring unit may be configured to switch a movement mode of the patient support apparatus to a safety mode as a function of a set grip position of the at least one handle element. In safety mode, a movement (e.g., an introducing movement of the patient support apparatus into a patient receiving region of a medical imaging apparatus) is prevented and/or blocked. This embodiment of the monitoring unit advantageously prevents an introducing movement of the patient support apparatus into the patient receiving region when the handle element is set such that it would result in a collision with a housing of the medical imaging apparatus enclosing the patient receiving region.

### BRIEF DESCRIPTION OF THE DRAWINGS

FIG. 1 shows a schematic diagram of one embodiment of a medical imaging apparatus with a patient support apparatus;

FIG. 2 shows a schematic diagram of one embodiment of a patient support apparatus; and

FIG. 3 shows a schematic diagram of a further exemplary embodiment of one embodiment of a patient support apparatus.

### DETAILED DESCRIPTION

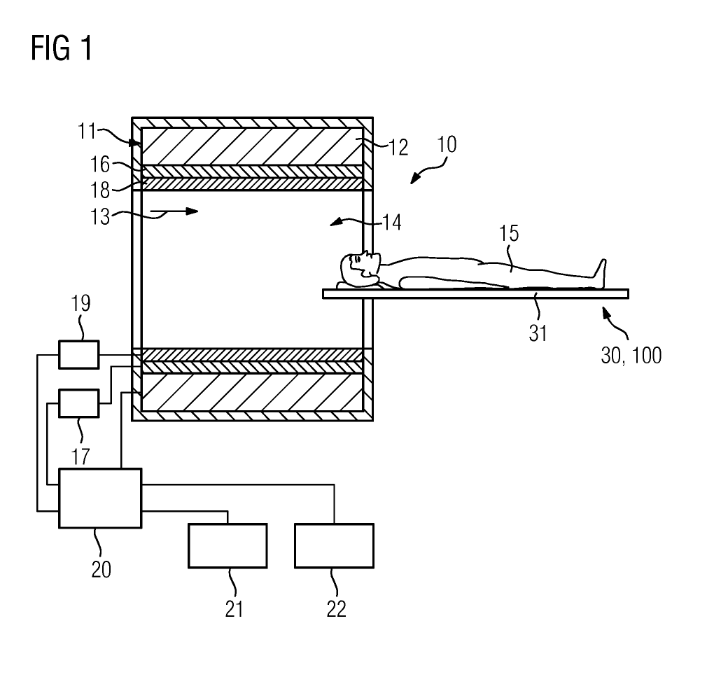

FIG. 1 shows a schematic diagram of one embodiment of a medical imaging apparatus. In the present exemplary embodiment, the medical imaging apparatus is formed by way of example by a magnetic resonance apparatus 10. Alternatively, the medical imaging apparatus may also include a computed tomography apparatus, a PET apparatus, a C-arm apparatus and/or further configurations of a medical imaging apparatus.

The magnetic resonance apparatus 10 includes a magnet unit 11 with a main magnet 12 for generating a powerful and, for example, constant main magnetic field 13. The magnetic resonance apparatus 10 also has a patient receiving region 14 for receiving a patient 15. In the present exemplary embodiment, the patient receiving region is configured as cylindrical and is enclosed in a cylindrical manner in a circumferential direction by the magnet unit 11. A different configuration of the patient receiving region 14 may also be provided. The patient 15 may be moved by a patient support apparatus 30 of the magnetic resonance apparatus 10 into the patient receiving region 14. The patient support apparatus 30 has a table 31 that is arranged in a movable manner within the patient receiving region 14 of the magnetic resonance apparatus 10.

The magnet unit 11 also has a gradient coil unit 16 for generating magnetic field gradients that are used for spatial encoding during imaging. The gradient coil unit 16 is controlled by a gradient control unit 17 of the magnetic resonance apparatus 10. The magnet unit 11 further includes a high-frequency antenna unit 18 and a high-frequency antenna control unit 19 for exciting a polarization that is established in the main magnetic field 13 generated by the main magnet 11. The high-frequency antenna unit 18 is controlled by the high-frequency antenna control unit 19 and radiates high-frequency magnetic resonance sequences into an examination space, which is essentially formed by the patient receiving region 14.

To control the main magnet 12 of the gradient control unit 17 and to control the high-frequency antenna control unit 19, the magnetic resonance apparatus 10 has a control unit 20 formed by a computation unit. The control unit 20 controls the magnetic resonance apparatus 10 centrally (e.g., performing a predetermined imaging gradient echo sequence). The control unit 20 also includes an evaluation unit (not shown in detail) for evaluating image data. Control information, such as imaging parameters, for example, and reconstructed magnetic resonance images may be displayed to an operator on a display unit 21 (e.g., on at least one monitor) of the magnetic resonance apparatus 10. The magnetic resonance apparatus 10 also has an input unit 22, by which information and/or parameters may be input by an operator during a measuring operation.

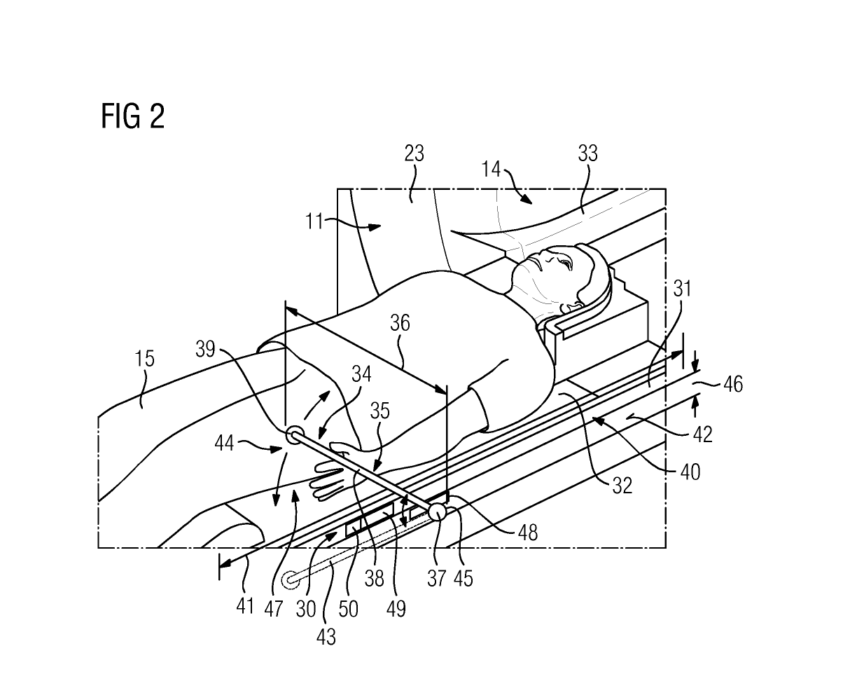

FIG. 2 shows one embodiment of a patient support apparatus 30 in more detail. The patient support apparatus 30 is configured to support the patient 15, for example, for a medical imaging examination using the medical imaging apparatus. The patient support apparatus 30 has the table 31 with a support surface 32 for supporting the patient 15. The patient support apparatus 30 also includes a base unit 33. The table 31 is configured so that the table 31 may be moved in relation to the base unit 33, allowing the table 31 to be introduced into the patient receiving region 14 of the medical imaging apparatus. In the present exemplary embodiment, the patient support apparatus 30 is integrated within the medical imaging apparatus, so the base unit 33 is arranged within the patient receiving region 14.

The patient support apparatus 30 also has a patient support assistance unit 34 with a handle element 35. The patient support assistance unit 34 (e.g., the handle element 35 of the patient support assistance unit 34) is configured to assist a support operation (e.g., a change of support operation and/or a repositioning operation from a sitting position to a horizontal position or from a horizontal position to a sitting position) for the patient 15. The handle element 35 of the patient support assistance unit 34 is, for example, configured so that the patient 15 may support him/herself with his/her hand on the patient support assistance unit 34 (e.g., on the handle element 35 of the patient support assistance unit 34) during a change of support and/or repositioning.

In the present exemplary embodiment, the patient support assistance unit 34 includes a single handle element 35. An embodiment of the patient support assistance unit 34 with two or more handle elements 35 may also be provided.

In the present exemplary embodiment, the handle element 35 is configured as a rod, and in a longitudinal extension 36 of the handle element 35, the handle element 35 has a first end region 37 that includes a support region. The handle element 35 also has a grip region 38 that, in the longitudinal extension 36 of the handle element 35, is arranged in a center region and in an end region 38 of the handle element 35 opposite an end region 37 including the support region.

The support region provides that the handle element 35 of the patient support assistance unit 35 is arranged in a peripheral region 40 of the table 31 (e.g., a peripheral region 40 of the support surface 32 of the table 31), so that any obstruction of a support operation for the patient 15 by the patient support assistance unit 34 (e.g., the handle element 35 of the patient support assistance unit 34) is prevented. Also, the handle element 35 is arranged in a center region of the table 31, so that the handle element 35 is easily accessible for a patient 15 lying on the table 31, or for a patient 15 being moved from a sitting position to a horizontal position or from a horizontal position to a sitting position. The center region is arranged in the center in the direction of and/or along a longitudinal extension 41 of the table 31.

In the present exemplary embodiment, the patient support assistance unit 34 (e.g., the handle element 35 of the patient support assistance unit 34) is arranged on a side 42 of the table 31. The side 42 is aligned perpendicular to the support surface 32.

In one alternative embodiment, the support surface 32 may have a cutout in a lateral peripheral region, and the patient support assistance unit 34 (e.g., the handle element 35 of the patient support assistance unit 34) may be arranged in the cutout.

The handle element 35 of the patient support assistance unit 34 is arranged in a movable manner on the table 31. The handle element 35 is arranged such that the handle element 35 may be moved between a first grip position 43 (shown with a broken line in FIG. 2) and at least one second grip position 44. In the present exemplary embodiment, the handle element 35 is supported on the table 31 such that the handle element 35 may be rotated between the at least two grip positions 43, 44. The patient support assistance unit 34 has a rotary bearing unit 45 that allows rotation of the handle element 35 about a rotation axis on the table 31. The rotation axis is aligned essentially parallel to the support surface 32 of the table 31 and essentially parallel to a transverse extension of the table 31. The rotary bearing unit 45 is arranged within the support region of the handle element 35.

In a first grip position 43, the handle element 35 is arranged in a passive grip position on the table 31. The handle element 35 is, for example, arranged almost completely within a thickness 46 of the table 31, so that the handle element 35 in this first grip position 43 does not project beyond the support surface 32 of the table 31 and does not extend into a patient support region 47 of the table 31.

When the handle element 35 is in a second grip position 44 on the table 31, the handle element 35 (e.g., the grip region 38 of the handle element 35) extends into the patient support region 47 of the table 31. The handle element 35, for example, projects beyond the support surface 32 of the table 31. The second grip position 44 of the handle element 35 includes an active grip position, in which support may be provided for the patient 15 at the handle element 35. In the second grip position 44, the longitudinal extension 36 of the handle element 35 is inclined at an angle of at least 45° to maximum 90° to the support surface 32 of the table 31.

To set a grip position 43, 44, the patient support assistance unit 34 and/or the table 31 has a setting unit 48. The setting unit 48 includes the rotary bearing unit 45 as well as a setting element (not shown in detail), by which one of the grip positions 43, 44 may be locked, so that the handle element 35 is secured in the set grip position 43, 44. The patient support assistance unit 34 may be configured such that the handle element 35 may be locked in different second grip positions 44 using the rotary bearing unit 45 and the setting unit 48.

If a patient 15 is on the support surface 32 of the table 31 in a sitting or horizontal position and the patient 15 is to be supported differently and/or repositioned (e.g., from the sitting position to a horizontal position or from the horizontal position to a sitting position), the handle element 35 is moved by the setting unit 48 and the rotary bearing unit 45 to a second grip position 44 on the table and secured in this second grip position 44. The patient 15 may grip the handle element 35 with one hand and support the patient 15 for the change of support operation and/or the repositioning operation using the handle element 35. This significantly reduces the force expenditure on the part of a medical operator (e.g., a physician or medical technical assistant).

If a medical imaging examination is performed using the medical imaging apparatus after the change of support and/or repositioning, the handle element 35 is to be moved beforehand from the second grip position 44 to the first grip position 43 using the setting unit 48 and the rotary bearing unit 45 and is to be secured similarly in this first grip position 43. Only when the handle element 35 is supported in the first grip position 43 on the table 31 may the table 31 be moved into the patient receiving region 14 of the medical imaging apparatus in order to prevent a collision of the patient support assistance unit 34 (e.g., the handle element 35 of the patient support assistance unit 34) with a housing 23 of the medical imaging apparatus enclosing the patient receiving region 14.

To detect and/or monitor a grip position 43, 44 of the handle element 35 on the support table 31, the patient support apparatus 30 has a monitoring unit 49. The monitoring unit 49 includes at least one sensor unit 50 configured to detect a position of the handle element 35 on the table 31. In this process, the monitoring unit 49 switches a movement mode of the patient support apparatus 30 (e.g., a movement mode of the table 31) to a safety mode as a function of a set grip position 44 of the handle element 35.

If the sensor unit 50 detects that the handle element 35 is in a second grip position 44 on the table 31, the monitoring unit 48 switches the patient support apparatus 30 (e.g., the table 31) to safety mode, so that movement of the patient support apparatus 30 (e.g., the table 31) is no longer possible. Safety mode includes a passive movement mode, so that movement of the patient support apparatus 30 (e.g., the table 31) is blocked.

If the sensor unit 50 detects that the handle element 35 is in the first grip position 43 on the table 31, the movement mode of the patient support apparatus 30 (e.g., the table 31) remains active, so that movement of the patient support apparatus 30 (e.g., the table 31) may be provided at any time. If the handle element 35 is moved from the first grip position 43 to a second grip position 44 during movement of the table 31, the monitoring unit 49 stops movement of the patient support apparatus 30 (e.g., the table 31) and deactivates the movement mode of the table 31.

The monitoring unit 49 is also configured such that a position of the patient support apparatus 30 (e.g., the table 31) in relation to the patient receiving region 14 of the medical imaging apparatus may also be detected, and a safety mode for movement of the handle element 35 of the patient support assistance unit 34 may be set accordingly. Thus, for example, the monitoring unit 49 detects whether the patient support apparatus 30 (e.g., the table 31) is within the patient receiving region 14 of the medical imaging apparatus. If so, the handle element 35 is locked in a first grip position 43 by the setting unit 48, so that the handle element 35 may not be moved out of the first grip position 43.

To detect and/or monitor a grip position 43, 44 of the handle element 35 on the support table 31, the monitoring unit 49 includes a processor unit (not shown in detail) and the required software and/or computer programs that are stored in a memory unit (not shown in detail) of the monitoring unit 49. The software and/or computer programs are configured to detect and monitor the positions of the handle element 35 together with the sensor unit 50 and also to switch a movement mode of the patient support apparatus 30 (e.g., the table 31) to safety mode as a function of the grip position 43, 44 of the handle element 35 on the table 31.

In an alternative embodiment, the monitoring unit 49 may also be arranged outside the patient support apparatus 30 but within the medical imaging apparatus (e.g., within a patient receiving region 14 of the medical imaging apparatus). The monitoring apparatus 49 may also be integrated at least partially within the control unit 20 of the medical imaging apparatus.

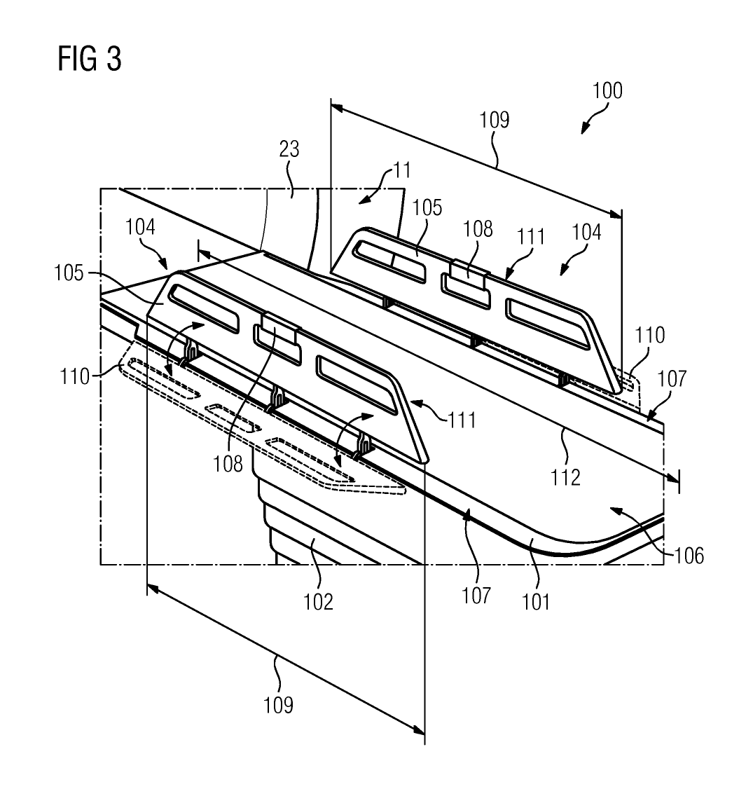

FIG. 3 shows an alternative exemplary embodiment of the patient support apparatus 100. Essentially same components, features and functions are identified with the same reference characters. The description that follows is essentially restricted to the differences compared with the exemplary embodiment in FIG. 2, reference being made to the description of the exemplary embodiment in FIG. 2 with respect to the same components, features and functions.

The patient support apparatus 100 is formed by a mobile patient support apparatus that may be moved independently of the medical imaging apparatus. The patient support apparatus 100 has a table 101 and a base unit 102. The table 101 includes a support surface for supporting and/or positioning the patient 15. The table 101 is also configured so that the table 101 may be moved in relation to the base unit 102, so the table 101 may be moved into the patient receiving region 14 of the medical imaging apparatus for a medical imaging examination.

The patient support apparatus 100 has a patient support assistance unit 104 that includes two boundary elements 105. The two boundary elements 105 are arranged on opposing sides to delimit a patient support region 106 of the patient support apparatus on the table. The boundary elements 105 are, for example, arranged in a peripheral region 107 of the table 101. The boundary elements 105 each have a handle element 108 that is arranged in a center region of the boundary elements 105. The center region is arranged in the center in the direction of and/or along a longitudinal extension 109 of the boundary elements 105.

The boundary elements 105 are supported in a movable manner on the table 101. The boundary elements 105 may be folded away to the side when not in use. The handle elements 108 are thus supported such that the handle elements 108 may be moved between two different grip positions 110, 111 on the table 101. The patient support assistance unit 104 has a rotary bearing unit 112.

The handle elements 108 are arranged in a central peripheral region of the table 101. The central peripheral region is arranged in the center in the direction of a longitudinal extension 112 of the table 101.

In a first grip position 110, the handle elements 108 are arranged essentially completely within a thickness of the table 101, so that the handle elements 108 do not extend into the patient support region 106 of the patient support apparatus 100. In a second grip position 111, the handle elements 108 are aligned essentially parallel to the longitudinal extension 112 of the table 101.

A further configuration of the patient support assistance unit 104 and a monitoring unit in FIG. 3 corresponds to the embodiments relating to the exemplary embodiment in FIG. 2.

Although the invention has been illustrated and described in detail using the exemplary embodiments, the invention is not limited by the disclosed examples. Other variations may be derived therefrom by the person skilled in the art without departing from the scope of protection of the invention.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A patient support apparatus configured to support a patient, the patient support apparatus comprising:
a table comprising a support surface to support the patient,
wherein the table comprises a patient support assistance unit with at least one handle element.

2. The patient support apparatus of claim 1, wherein the at least one handle element is arranged at least partially in a center region of the table.

3. The patient support apparatus of claim 1, wherein the at least one handle element is arranged in a peripheral region of the table.

4. The patient support apparatus of claim 1, wherein the at least one handle element is arranged on a side of the table, the side being aligned perpendicular to the support surface.

5. The patient support apparatus of claim 1, wherein the at least one handle element is supported on the table such that the at least one handle element is movable between a first grip position and at least one second grip position.

6. The patient support apparatus of claim 1, wherein the table has a thickness, and the at least one handle element is arranged on the table within the thickness in a grip position.

7. The patient support apparatus of claim 1, wherein the at least one handle element extends into a patient support region of the table in a grip position on the table.

8. The patient support apparatus of claim 7, wherein the at least one handle element has a longitudinal extension that is inclined at an angle between at least 45° and maximum 90° to the support surface of the table in the grip position on the table.

9. The patient support apparatus of claim 7, wherein the at least one handle element has a longitudinal extension, the longitudinal extension of the at least one handle element being aligned essentially parallel to a longitudinal extension of the table when the at least one handle element is in the grip position on the table.

10. The patient support apparatus of claim 1, wherein the table, the patient support assistance unit, or the table and the patient support assistance unit include a setting unit configured to set a grip position of the at least one handle element on the table.

11. The patient support apparatus of claim 1, further comprising a sensor unit operable for detecting at least one grip position of the at least one handle element on the table.

12. A medical imaging apparatus comprising:
a magnet unit;
a patient receiving region at least partially enclosed by the magnet unit; and
a patient support apparatus configured to support a patient, the patient support apparatus comprising:
a table comprising a support surface to support the patient,
wherein the table comprises a patient support assistance unit with at least one handle element.

13. The medical imaging apparatus of claim 12, further comprising a monitoring unit configured for monitoring a grip position of the at least one handle element of the patient support assistance unit.

14. The medical imaging apparatus of claim 13, wherein the monitoring unit is configured to switch a movement mode of the patient support apparatus to a safety mode as a function of a set grip position of the at least one handle element.

15. The medical imaging apparatus of claim 13, wherein the monitoring unit comprises a sensor unit.

16. The medical imaging apparatus of claim 12, wherein the at least one handle element is arranged at least partially in a center region of the table.

17. The medical imaging apparatus of claim 12, wherein the at least one handle element is arranged in a peripheral region of the table.

18. The medical imaging apparatus of claim 12, wherein the at least one handle element is arranged on a side of the table, the side being aligned perpendicular to the support surface.

19. The medical imaging apparatus of claim 12, wherein the at least one handle element is supported on the table such that the at least one handle element is movable between a first grip position and at least one second grip position.

20. The medical imaging apparatus of claim 12, wherein the table has a thickness, and the at least one handle element is arranged on the table within the thickness in a grip position.
